(19) United States
(12) Patent Application Publication
(10) Pub. No.: US 2021/0239667 A1
(43) Pub. Date: Aug. 5, 2021

(54) SHOULDER MOUNTABLE REAL-TIME AIR QUALITY MEASUREMENT DEVICE AND AIR QUALITY DEVICE CALIBRATION SYSTEM

(71) Applicant: YALE UNIVERSITY, New Haven, CT (US)

(72) Inventors: Drew Gentner, New York, NY (US); Fulizi Xiong, New Haven, CT (US)

(21) Appl. No.: 17/269,825

(22) PCT Filed: Aug. 20, 2019
(86) PCT No.: PCT/US19/47129

§ 371 Date: Feb. 19, 2021

Related U.S. Application Data
(60) Provisional application No. 62/719,806, filed on Aug. 20, 2018.
(60) Provisional application No. 62/756,373, filed on Nov. 6, 2018.

(51) Int. Cl. G01N 33/00; G01N 1/22; G01N 21/27

(52) U.S. Cl. CPC G01N 33/0006; G01N 1/2273; G01N 21/274; G01N 2001/2276; G01N 33/0031; G01N 2201/0221; G01N 1/2205

(57) ABSTRACT

An air quality measurement device includes a housing configured to rest on a shoulder of a user. The housing includes an air inlet directed to a breathing zone of the user, and an air outlet. An air quality sensor and an air pump within the housing is connected in-line between the air inlet and the air outlet. A calibration system for an air quality measurement device includes a gas sensor, a particulate matter sensor, and calibration elements: a particulate matter zeroing element configured to calibrate the particulate matter sensor, a gas sensor zeroing element to calibrate the zero response of the gas sensor(s), and a gas sensor known concentration element to calibrate the concentration response of the gas sensor(s).

### CROSS-REFERENCE TO RELATED APPLICATIONS

This application claims priority to U.S. Provisional Patent Application Nos. 62/719,806, filed on Aug. 20, 2018, and 62/756,373, filed on Nov. 6, 2018, both of which are incorporated herein by reference in their entireties.

### BACKGROUND OF THE INVENTION

Low-cost air pollutant devices are known to be very problematic in terms of their changes in zero, response factors, and sensitivity to relative humidity and temperature effects on zero or response factors. This has led to decreased confidence in their data for research or monitoring purposes, with the common calibration solutions being repeated visits to calibrate the monitors in the field using a gas cylinder or other device, or the periodic removal of the device for calibration/characterization in the lab. This considerably decreases the utility and feasibility of large distributed networks of monitors for high spatiotemporal resolution measurements of pollution in urban or non-urban areas, which is a major area of high-priority research around the globe.

Further, human exposure to air pollution is determined by the air pollutants that are present in the breathing zone, but measurements from this region are difficult, especially without being too obtrusive. In many current applications, people are required to wear large and often heavy backpacks (reducing compliance) that contain measurement equipment with tubing going up to their breathing region. Long tubing lines are problematic since they can lead to losses of small particles (that are naturally charged) or reactive pollutants to the tubing walls in the short time necessary to transport the pollutants down to the instruments.

While low-cost pollutant measurement devices are very attractive given their potential for deploying a wide range of monitors in a study area, their measurements are typically limited by the need for calibration devices that are considerably more expensive and much larger than the devices themselves (e.g. large gas cylinders, regulators, zero air generation systems, pollutant removal devices with catalysts). Accurate high spatiotemporal resolution measurements of air pollutants in large networks of sensors are difficult because the low-cost sensors are especially prone to drift, unit-to-unit variability, and other changes in their calibration over time, such as responses to changes in relative humidity and temperature. Current fixes are occasional site visits to calibrate units in the field with a cylinder or reference units or clustering multiple low-cost units together. These methods are inefficient in either personnel time or cost, and still result in only marginal gains with infrequent calibration.

Exposures to air pollution are associated with elevated health risks such as cardiorespiratory inflammatory responses and oxidative stress. Each year outdoor air pollution leads to approximately 3.3 million premature deaths worldwide. Assessment of public health risks and regulatory standards requires accurate measurement of air pollution levels. However, the traditional analytical techniques for air pollutant measurements, such as spectroscopy, chemiluminescence, and mass spectrometry, are expensive, which limits the deployment of instruments to sparsely located state and local air quality monitoring sites. As a result, the spatiotemporal variations of urban exposure caused by local traffic and individual point sources are not well characterized, calling for intra-urban monitoring with denser environmental observation networks.

While low-cost sensors have great potential to provide air quality data at higher spatiotemporal resolution and complement existing monitoring sites, multiple studies have reported measurement biases caused by sensor drift due to environmental variables and aging. Hence, careful sensor characterization, calibration and data processing are important to ensure measurement accuracy.

What is needed in the art is a personal, portable pollution monitoring device that allows for measurements of a wide range of important air pollutants in the breathing zone that is unobtrusive and does not require the use of a backpack. There is also a need for an improved calibration system and method that can be used to calibrate small and low-cost measurement devices. The present invention satisfies this need.

### SUMMARY OF THE INVENTION

In one embodiment, an air quality measurement device includes a housing configured to rest on a shoulder of a user including an air inlet directed to a breathing zone of the user, and an air outlet; an air quality sensor and an air pump within the housing and connected in-line between the air inlet and the air outlet. In one embodiment, the housing comprises a form-fitting crescent shape. In one embodiment, the housing comprises an attachment mechanism configured to attached to a shoulder area of apparel or a shoulder strap. In one embodiment, the shoulder strap is one of a bag strap, a backpack strap and a harness strap. In one embodiment, the air inlet is directed towards an area in front of a user's face and the air outlet is directed to an area behind the user. In one embodiment, the air quality sensor is at least one of a gas sensor and a particulate matter sensor. In one embodiment, the device includes a connection to an external battery and a cell board. In one embodiment, an air quality measurement system includes the device and a battery and cell board unit configured to attach to a user using an attachment mechanism separate from the housing.

In one embodiment, a calibration system for an air quality measurement device includes a gas sensor, a particulate matter sensor, and particulate matter zeroing element configured to calibrate the particulate matter sensor. In one embodiment, the system includes an outlet to a manifold housing the gas sensor, connected to the particulate matter zeroing element. In one embodiment, the system includes a 3-way valve configured to switch airflow from the outlet between the particulate matter zeroing element and a particulate matter sensor inlet. In one embodiment, the system further comprises a gas phase zeroing element comprising a packed bed of mixed catalysts and/or adsorbents configured to filter out pollutants. In one embodiment, the packed bed comprises at least one of soda lime, ascarite, activated carbon, molecular sieves and steel wool. In one embodiment, the packed bed comprises at least two of soda lime, ascarite, activated carbon, molecular sieves and steel wool. In one embodiment, the packed bed comprises at least three of soda lime, ascarite, activated carbon, molecular sieves and steel wool. In one embodiment, the packed bed comprises soda lime, ascarite, activated carbon, molecular sieves and steel wool. In one embodiment, the gas phase zeroing element comprises a cylinder comprising pure air or air with zero concentration of the measured pollutant (and cross-responsive pollutants).

In one embodiment, the gas phase known concentration calibration element comprises a cylinder comprising a gas standard. In one embodiment, the gas phase calibration element comprises a UV-generating lamp configured to generated a constant concentration of ozone. In one embodiment, the system is configured to provide known concentration calibration measurements across a range of relative humidity and temperature points. In one embodiment, the system is configured to provide zero calibration measurements across a range of relative humidity and temperature points. In one embodiment, the system further comprises a water vapor permeation device configured to maintain a substantially constant relative humidity inside the manifold. In one embodiment, the system is configured to provide air quality measurement for a plurality of pollutants.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A is a perspective view of a shoulder mountable real-time air quality measurement device according to one embodiment;

FIG. 1B is an alternate perspective view of a shoulder mountable real-time air quality measurement device according to one embodiment;

FIG. 2A and FIG. 2B are views of a shoulder-mountable real-time air quality measurement device according to another embodiment;

FIG. 3 is a view of a stationary real-time air quality measurement device with an included calibration system according to one embodiment;

FIG. 4 is a diagram of a calibration system according to one embodiment;

FIG. 5 is two photographs of a stationary air quality measurement device according to one embodiment;

FIG. 6 is a simplified electrical and flow diagram of an air quality measurement device according to one embodiment;

FIG. 7 is a photograph of a prototype stationary air quality measurement device according to one embodiment;

FIG. 8 is a photograph of a prototype stationary air quality measurement device according to one embodiment;

FIG. 9 is two photographs of a prototype stationary air quality measurement device according to one embodiment;

FIG. 10 is a real-time data tracking interface according to one embodiment;

FIG. 11 is a set of graphs showing outdoor data related to an air quality measurement device compared to reference data collected via government instrumentation;

FIG. 12 is a manifold for use with a multi-pollutant monitoring device according to one embodiment;

FIG. 13 is a graph of zero calibration data for a PM sensor using the PM zeroing channel;

FIG. 14 is a graph of experimentally measured data from a multi-pollutant monitoring device;

FIG. 15 is a graph of experimentally measured data from a multi-pollutant monitoring device;

FIG. 16 is a graph of experimentally measured data from a multi-pollutant monitoring device;

FIG. 17 is a graph of experimentally measured data from a portable multi-pollutant monitoring device;

FIG. 18 is a graph of experimentally measured data from a portable multi-pollutant monitoring device and a map indicating where measurements were taken;

FIG. 19A and FIG. 19B are graphs of correlation data from multiple co-located air quality monitoring devices;

FIG. 20 is a graph of air concentration data response time from air quality sensors;

FIG. 21 is two graphs of example data from calibration system operation for air quality measurement devices; and

FIG. 22A and FIG. 22B are graphs of outdoor data for air quality measurement devices and a comparison to reference measurements.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in air quality measurement devices and calibration systems. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G or 4G/LTE networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

Some aspects of the present invention may be made using an additive manufacturing (AM) process. Among the most common forms of additive manufacturing are the various techniques that fall under the umbrella of “3D Printing”, including but not limited to stereolithography (SLA), digital light processing (DLP), fused deposition modelling (FDM), selective laser sintering (SLS), selective laser melting (SLM), electronic beam melting (EBM), and laminated object manufacturing (LOM). These methods variously “build” a three-dimensional physical model of a part, one layer at a time, providing significant efficiencies in rapid prototyping and small-batch manufacturing. AM also makes possible the manufacture of parts with features that conventional subtractive manufacturing techniques (for example CNC milling) are unable to create.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is an air quality measurement device and calibration system.

Embodiments of the shoulder mountable real-time air quality measurement device described here are automated, small, low-cost, and improve the realism of measurements for one or multiple pollutants by measuring directly from the breathing zone to get better measurements of human exposure. Embodiments of the calibration system improve the accuracy of measurements and devices can be used to correct for several major issues in low-cost measurement devices, including but not limited to: zero concentration instrument response (i.e. “zeros”), calibration response factors, and their sensitivity to relative humidity and temperature. Embodiments of the devices also use chemical cross-response (i.e. cross sensitivity) on other detectors to calibrate for more pollutants than are included in the small cylinder. Since the relative humidity resulting from the calibration elements and the device temperature changes with environmental conditions, it provides zero measurements across a range of relative humidity and temperature points that allow the user to determine zeros and response factors to relative humidity and pressure and correct their measurements. Accordingly, embodiments described herein enable better measurements of a wide range of pollutants that cause many of the health effects associated with air pollution.

In some embodiments, a portable device of the present invention may include tethering functionality to other mobile devices, for example a smartphone or tablet computer. A wireless communication link to a smartphone or other portable electronic device may provide a means of data logging and transmission, as well as methods of collating the collected data with additional sensors present in the portable electronic device, for example a GPS, accelerometer, gyroscope, etc.

Advantageously, embodiments of the device are able to make accurate measurements of the concentrations of pollutants that the user is actually exposed to (rather than elsewhere on the body or at the closest stationary sampling site) with pollutant measurement sensors located on the shoulder. Embodiments of the device do not require a backpack and are small and easily mounted to the shoulder. Advantageously, embodiments of the device are configured such that all air measurement and monitoring elements are disposed within a single housing, the housing being completely mountable on the shoulder of a user. Also, embodiments of the device are able to measure a wide range of pollutants, alone or in combination, including but not limited to particulate matter (sizes including but not limited to PM10, PM2.5, and PM1), ozone, carbon monoxide, carbon dioxide, nitrogen dioxide, sulfur dioxide and nitric oxide. Low-cost pollutant measurement devices according to the embodiments described herein offer significant advantage over conventional devices, and can be provided at a lower cost, yielding the potential for increasing the accessibility of devices which accurately measure pollution. They are also smaller, more portable, and comfortable, providing an improved cost-effective tool for various entities including citizens, research institutions, companies monitoring OSHA exposure, and governmental agencies.

Some embodiments of the present invention refer to networks of pollution monitoring devices, that may comprise portable multipollutant monitors, stationary multipollutant monitors, or a combination thereof. Networks of such devices increase access to research-quality data that can be employed by researchers and policy-makers in industry, academia, and regional, national, and international agencies—all with data of unprecedented spatiotemporal and chemical resolution that is extremely powerful to decipher the inherent complexity of air pollution problems across chemical, spatial, temporal scales.

Embodiments of a pollution measurement device calibration system and architecture described herein allows for calibration of small, low-cost measurement devices. Embodiments of these devices enable users to frequently (e.g. multiple times a day or week) calibrate their sensors remotely and automatically. The embodiments are relatively simple and can integrate with existing microcontroller systems (e.g. Arduino).

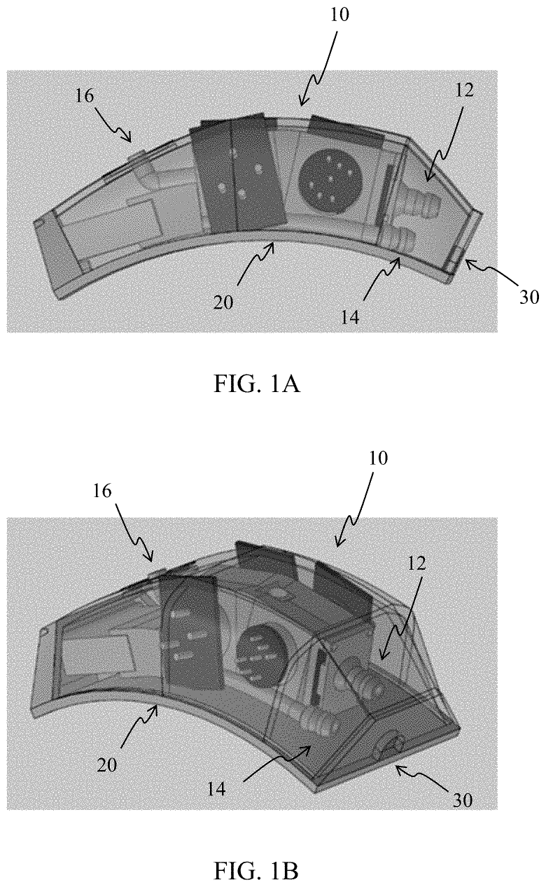

With reference now to FIGS. 1A and 1B, in one embodiment, a shoulder mountable real-time air quality measurement system includes components found in air quality measurement devices, such as batteries, processing board(s), and one or more sensors or microsensors. Sensors and pumps are mounted on the shoulder device 10 near the user's breathing zone (i.e. nose/mouth). The device 10 includes a housing 20 that is shaped or adjustable to mount directly on the shoulder or onto another object, such as apparel or a shoulder strap. The housing 10 includes openings 30 to allow air to run through the system for detecting air quality. Air inlets 12, 14 can be positioned near the user's inhalation breathing area, and exhaust outlets 16 configured away from this area, for example aimed behind the user's head.

In one embodiment, the shoulder device 10 is a clip on for an existing shoulder device that includes a shoulder strap (i.e. a purse, backpack, holster, or other shoulder strap), or it can be mounted to shoulder harness style straps, similarly to ones used by “GoPro” portable video recorders. In one embodiment, the monitor is attached to a harness that the user wears under their clothes. The physical shape of the shoulder device external housing can take multiple forms, as will be apparent to those having ordinary skill in the art. In one embodiment, a form-fitting crescent rests over the shoulder and is more inconspicuous. In other embodiments, the external housing takes the shape of a raised pod that is more prominent. In some embodiments, in the place of a single small housing positioned on one shoulder of a user, components of the device may be spread across two smaller housings positioned on either shoulder of the user. In some such embodiments, the two housings may be connected to one another, while in other embodiments they are not.

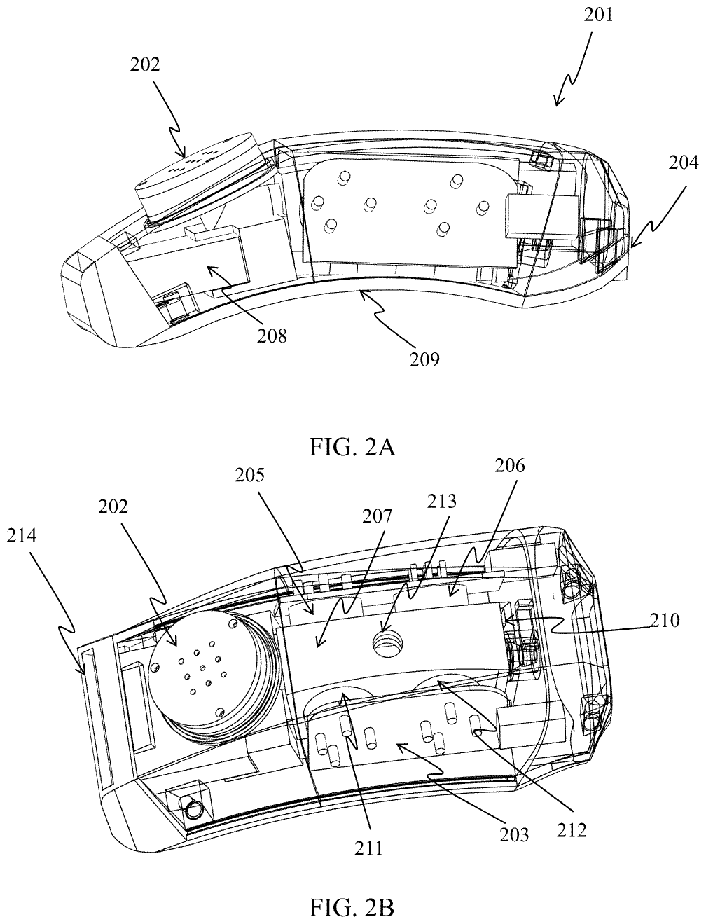

With reference now to FIG. 2A and FIG. 2B, another exemplary shoulder-mounted multi-pollutant monitoring device 201 is shown. The depicted shoulder device includes a gas-phase sample intake inlet 202 made of an inert material (e.g. PTFE) and containing a filter which may also be made of PTFE for preventing PM from entering the gas-phase channel and minimizing losses of reactive gas-phase pollutants (e.g. ozone). The filter is also configured to collect PM samples for more detailed offline chemical analysis. The device 201 also includes circuit boards 203, and outlet 204 for exhaust tubing and any necessary wiring. Air inlet 202 channels air into inert manifold 207, which is fluidly connected to one or more pollutant sensors 205, 206, 211, and 212. In some embodiments, inert manifold 207 has a very small internal volume for quick delivery of pollutants from the outside air to the sensors 205, 206, 211, and 212. The external housing 209 may in some embodiments include connection points, for example to easily attach to shoulder straps. Embodiments of a shoulder-mounted monitoring device may be mounted to a user's backpack, purse, bag, harness, or shoulder. In some embodiments, some or all parts of the shoulder-mounted monitoring device may be produced by additive manufacturing. In one embodiment, the external housing is produced by 3D printing.

The device may further include a small pump 210, fluidly connected to and configured to draw air through the manifold 207. In some embodiments, the manifold 207 includes an insert point 213 for one or more environmental sensors, for example a relative humidity and/or temperature sensor, which may be used to correct for any sensor variations due to environmental conditions.

In some embodiments, the device includes a dedicated inlet 214 for the PM sensor 208. The dedicated inlet 214 may include a light shield to protect the optical components of the PM sensor from interference, while also reducing any losses of PM due to impaction in the inlet. In some embodiments, the sensor 208 is placed right behind the inlet 214 to reduce losses inside the instrument. The PM sensor may include an internal pump and/or an exhaust channel that directs the exhaust to the back of the device. In some embodiments, the dedicated exhaust is incorporated into the manifold 207.

In one embodiment, rechargeable and/or disposable batteries and a cell board are attached to one of the lower straps (or placed in the user's existing bag) and all the power and communication lines could be bundled in a cable and run up to the shoulder where the housing holds the remainder of the elements of the device, including but not limited to sensors, small boards, and pumps, for example in a custom printable housing. In some embodiments, the battery and cell board are incorporated into the shoulder mounted device, alleviating the need for a secondary housing. In one embodiment, the housing conforms to the shoulder, or alternatively includes adjustable attachments (e.g. a 360 degree ball joint). To collect additional data, a camera can be mounted to the user's chest to acquire real-time footage that could be connected with their air quality data. The collection unit can be connected to users' smart phones to log data.

In one embodiment, some or all of the parts of a device of the present invention are produced via additive manufacturing. Any additive manufacturing process known in the art may be used, and in some embodiments parts of a device of the present invention may be fabricated with an additive manufacturing process in such a way that they would not be reasonably manufacturable using conventional subtractive manufacturing. In one embodiment, all the elements of a device of the present invention may be assembled into a further minimized shoulder mounted device. In one embodiment, all the elements of a device of the present invention may be assembled into a different form-fitting, or otherwise structurally-advantageous, housing. Further miniaturization is contemplated, to the size of a wearable pendant or necklace.

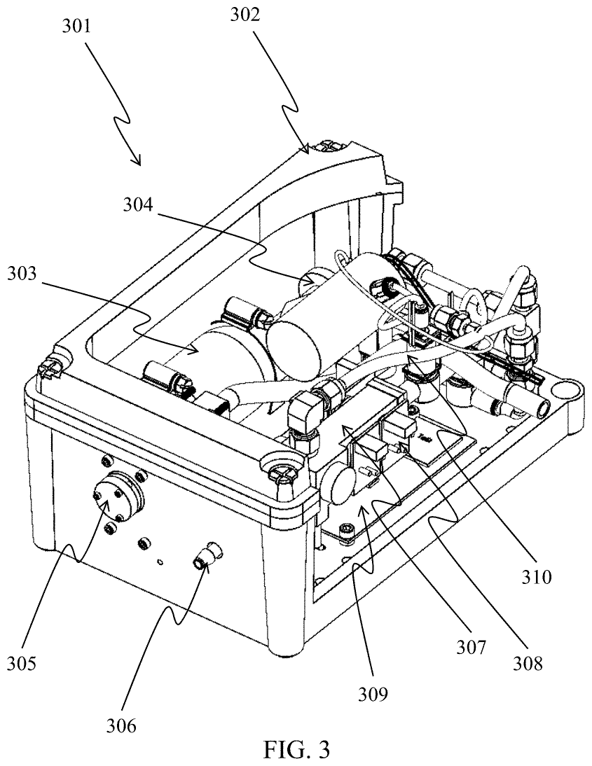

With reference now to FIG. 3, a stationary multi-pollutant monitor 301 is shown. The depicted stationary monitor is assembled into a weatherproof housing 302, for example a polycarbonate housing. The monitor includes a small-volume gas cylinder 303 and valve 304, and inlets for PM measurement (305) and gas phase (306). The PM inlet 305 may be electrically grounded to prevent losses of charged particles and to screen dust, and to allow for free flow of PM into the sensor. The gas phase inlet 306 may include a low-profile PTFE filter holder and PTFE filter positioned upstream from the manifold 307. Manifold 307 is leak-tight and fluidly connected to one or more sensors 308. The sensors 308 are electrically connected to daughter boards, which are in turn electrically connected to a main control board 309 including a cellular module or other communication circuitry. In some embodiments, the manifold 307 is made from an inert material. Stationary monitors may be used individually or within networks with other stationary monitors and/or portable monitors as contemplated herein. In some embodiments, stationary monitors are configured to measure and collect data on a wide variety of pollutants, including but not limited to size-resolved particulate matter, ozone, NO₂, NO, SO₂, CO, CO₂, CH₄. Stationary monitors like the monitor shown in FIG. 3 may be designed for long-term stationary use with minimal, infrequent maintenance by the user.

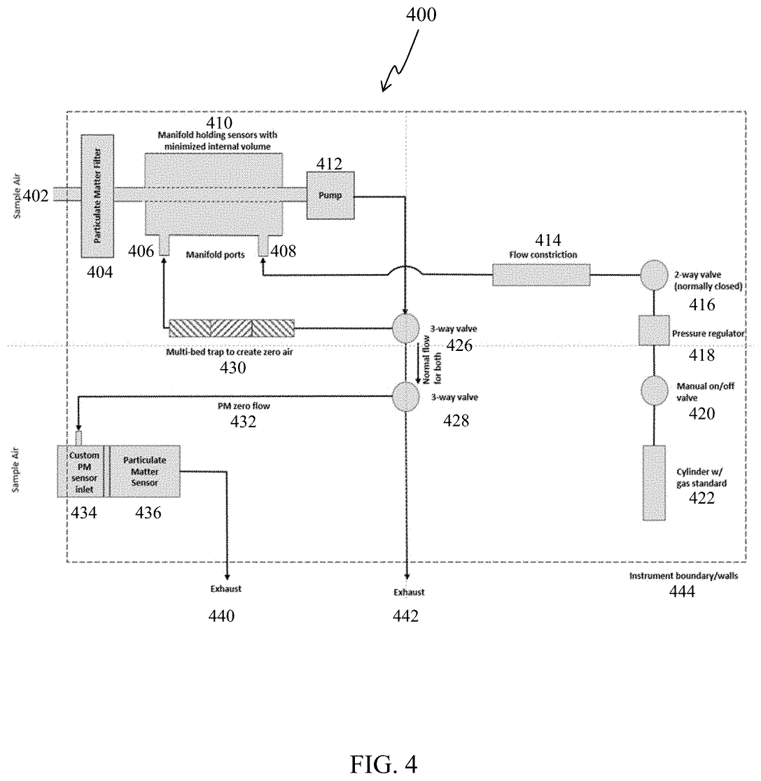

With reference now to FIG. 4, a calibration system according to one embodiment is shown. The system diagram shows the implementation of the system 400 to calibrate (zero and known concentration check) a mix of gas sensors that are in the “manifold”. In addition to the potential to “zero” the gas sensors in 410, there is “zeroing” capability for the particulate matter sensor 436. Incoming air in the manifold is pre-filtered of particles and with the switch of a valve 420 can provide a zero flow for the particle sensor 436, which is separate from the others due to the need to prevent particles from contaminating the gas sensors 410. The system 400 can include 1-2 very small lightweight gas cylinder(s) 422 that are filled with a mixture of calibration gases (that the device measures) at accurate concentrations. Another cylinder with pure “zero” air can be used for calibration, or can have a packed bed (in a tube) 430 of mixed catalysts and adsorbents (e.g. soda lime, ascarite, activated carbon, molecular sieves, steel wool, or other oxidizing or reducing materials) that can filter out the pollutants measured. In some embodiments, a small UV-generating lamp (housed for example in a sealed teflon plumbing tee) is used to generate a constant concentration of ozone for the gas flowing into the gas sensor housing. In some embodiments, a solid material that releases or produces a calibrant gas could be used to calibrate a sensor.

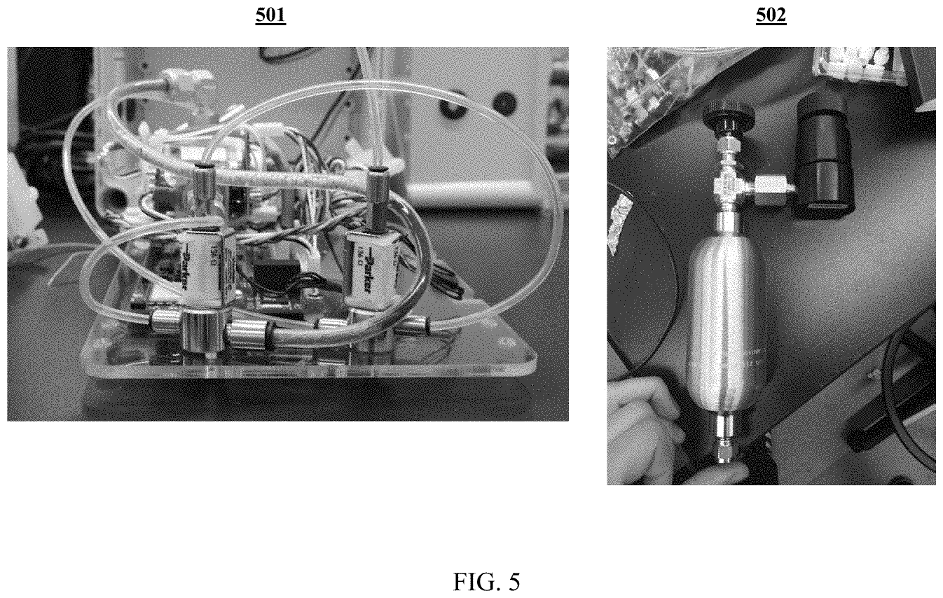

The system 400 can use these tools to automatically calibrate multiple times a day, every day, or every few days. It can be applied to a broad range of pollutant measuring devices, with applicability for anything that can be stably stored in a cylinder or similar container, or also for devices that will respond due to cross-interference to a different chemical compound (i.e. where a detector for a first compound will respond to high concentrations of a second compound that is contained in a standard mixture). An exemplary zero calibration system, showing valves and zero trap, is shown in photograph 501 in FIG. 5. An exemplary miniature calibration cylinder and regulator is shown in photograph 502 of FIG. 5.

In one embodiment, a zeroing element of the present invention may be used with one or more humidity and temperature sensors to more accurately calculate variations in response factors for temperature and humidity of various sensors (a common issue). In one embodiment, a measurement device of the present invention may obtain multiple zero measurements during the course of a single day, recording the temperature and humidity at the time of each measurement, in order to more accurately characterize one or more sensors of the present invention. In some embodiments, a measurement device of the present invention may use active cooling, heating, humidification, or dehumidification means in order to induce changes in temperature and/or humidity and obtain measurements from sensors under different conditions. In some embodiments, active humidification is used for certain sensors that require a certain amount of moisture in the air in order to function, where a measurement device containing such a sensor is placed in a location that sometimes reaches zero or near-zero relative humidity.

In some embodiments, a measurement device of the present invention comprises a permeation device for water, for example a thin film of permeable material configured to allow water to move across the interface at a slow rate to raise the relative humidity of the calibration gas. In some embodiments, a measurement device of the present invention may include a gas flow moving past a permeable membrane, or one or more diffusion or effusion devices, including but not limited to a constriction or pinhole, configured to provide a controlled rate of gas transfer across a small distance.

Certain embodiments of measurement devices of the present invention include a particulate matter monitoring channel and a separate gas phase channel fluidly connected to the aerosol channel but including a filter that removes particulate matter. For example a quantity of intake air may first pass through the particulate matter channel, then through a filter that removes the particulates, leaving only gas for sensing in a gas phase channel. In some embodiments, the filtered air from the gas phase channel may be pumped back into the particulate matter channel for use as a zeroing reference. Such a closed-loop configuration is advantageously efficient and minimizes flow volume.

In some embodiments, a multipollutant monitoring device of the present invention includes a very small internal volume for the manifold that holds the sensors and exposes them to a vacuum flow of air. Certain embodiments include a pump that can be temporarily turned off with a specially situated port that back flushes the standard for a short duration that is necessary to fully flush the chamber (e.g. 4 e-folding lifetimes or exchange volumes). In some embodiments, a calibration system includes a multi-bed packed tube for producing a zero concentration of the pollutants being measured that is designed to filter out the mix of pollutants for which the device is being calibrated. In some embodiments, a calibration system includes a small cylinder that is hooked up to a regulator with a critical orifice (narrow diameter tubing) to precisely meter flow with a valve, where the system precisely times the duration to open the valve.

With reference to FIG. 4, in one embodiment, the system 400 includes a manifold holding sensors with minimized internal volume 410 that connects to sample air 402 through a particulate matter filter 404. The manifold holding sensors 410 can have a first 406 and second 408 port. The second port 408 connects in-line to a flow constriction device 414, a 2-way valve 416, a pressure regulator 418, a manual valve 420 and a cylinder containing a gas standard 422. The first port 406 connects in-line to a multi-bed trap to create zero air 430 and a first 3-way valve 426. In one direction, the first 3-way valve 426 is connected to the manifold holding sensors 410 via a pump 412. In another direction, the first 3-way valve 426 is connected to a second 3-way valve 428. The second 3-way valve 428 connects to an exhaust port 442 in one direction, and a particulate matter sensor inlet 434 in another direction. The valves used in devices of the present invention may be any suitable valves known in the art, and may be electrically actuated or use some other actuation means. The particulate matter sensor inlet 434 leads to a particulate matter sensor 436, which then leads to an exhaust port 440. Communication with sample air via system inlets and outlets is provided by openings in the instrument housing 444.

Advantageously, the system 400 combines unique features to provide accurate and cost-efficient results. The manifold 410 that holds the sensors and exposes them to a forced flow of air is implemented with a small internal volume. The pump 412 can be temporarily turned off with a specially situated port that back flushes the standard for a short duration. This is necessary to fully flush the chamber (i.e. 4 e-folding lifetimes). The multi-bed packed tube 430 produces a zero concentration of the pollutants being measured that is designed to filter out the mix of pollutants that are being calibrated for. A multi-bed trap of the present invention advantageously includes a plurality of beds each made from different materials, each configured to remove, absorb, or adsorb a different compound or set of compounds. For example, a first bed in a multi-bed trap may be configured to remove NO, while a second bed might be configured to remove CO. A third bed may be configured to remove CO₂. Different beds may be made of a variety of reducing or oxidizing materials. In some embodiments, multiple beds each configured to perform a chemical reaction in sequence may be used. For example, a first bed may be configured to undergo a chemical reaction with a first compound, yielding a second compound, while a second bed may be configured to isolate, filter, or otherwise remove the second compound. Additional beds may be added to the chain, allowing for more complete absorption of the desired compounds. Although the listed configurations of beds are included here, it is understood that further combinations of beds and materials are also contemplated.

The small cylinder 422 is connected to a pressure regulator 418 with a critical orifice (narrow diameter tubing) to precisely meter flow with a valve and precisely times the duration to open the valve. Advantageously, the system includes calibration approaches for both the gas-phase sensors 410 and particulate matter sensor 436. Calibration can include sensor input fed to a mobile or cloud-based system that reads measurements for analyzing system calibration, and provides feedback to the system to make adjustments. Software that utilizes strategic guessing or machine learning can be implemented to determine the state and performance of the system, and make or suggest any adjustments.

Data may be logged from the monitors disclosed herein using any suitable data type or logging frequency. In some embodiments, data from some or all sensors may be logged at 1 Hz, 2 Hz, 5 Hz, or more. In other embodiments, data from some or all sensors may be logged at a slower rate, for example once per minute, once per five minutes, once per ten minutes, or once per fifteen minutes.

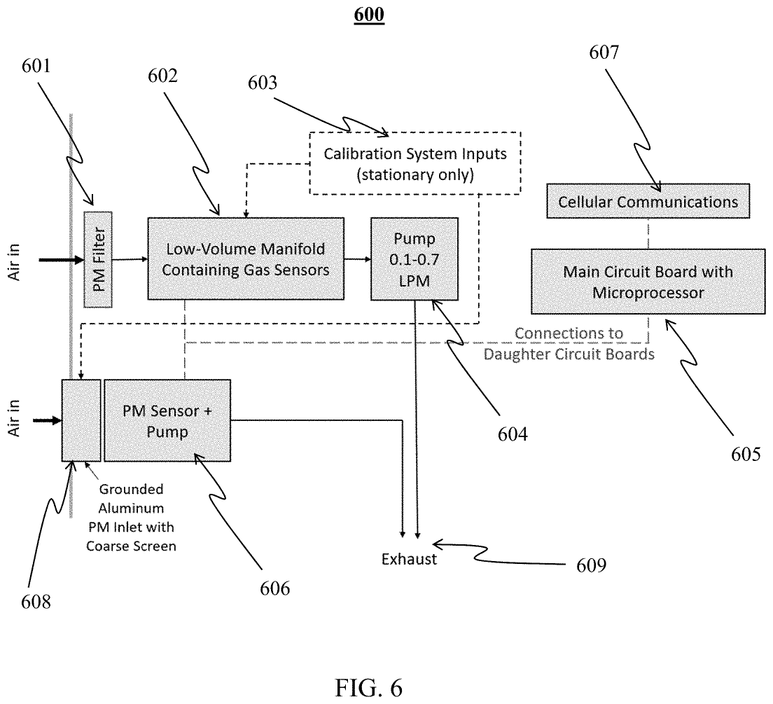

An exemplary simplified flow and electronics diagram of a multi-pollutant monitor of the present invention is shown in FIG. 6. System 600 includes two air intake paths, the first through PM filter 601 and the second through a grounded inlet 608 with a coarse screen for large (i.e. dia >10 μm) dust. The first intake path passes through a low-volume manifold containing gas sensors 602, driven by pump 604 out through exhaust 609. The second air intake path is driven through PM sensor+pump 606 through exhaust 606. The main circuit board 605 is electrically connected to the gas sensors in manifold 602 and sensor pump 606. The main circuit board 605 is additionally connected to cellular communications module 607, which is used at least for communicating recorded data back to a central server. Stationary multi-pollutant monitors may additionally include a calibration system 603 having inputs connected to the manifold gas sensors and second air inlet path 608.

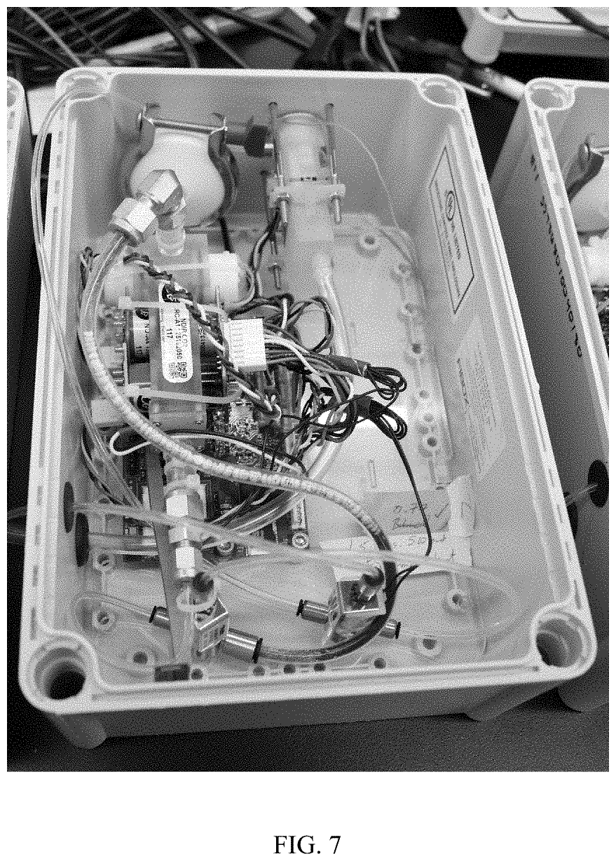

An exemplary stationary multi-pollutant monitor prototype is shown in FIG. 7. The depicted stationary monitor has a dedicated channel for gas and a dedicated channel for particulate measurements. The PM inlet is specially designed with a custom inlet and housing to reduce particle loss resulting from an electrically-charged inlet (inlet is electrically-grounded) or particle impaction. Gas sensors are kept in a separate housing that follows a custom low-profile PTFE filter holder, PTFE tubing and a custom 3-D printed inert sensor manifold with sensors arranged with most reactive gases measured near the front of the manifold. The system also contains the zero calibration system described elsewhere in this disclosure. The depicted exemplary monitor includes sensors for size-resolved particulate matter (mass and number concentration measurements) as well as various pollutant gases. The enclosure may be made of any suitable material, and in some embodiments may comprise polycarbonate or other weather-proof or weather-resistant materials. In some embodiments, the enclosure is entirely or substantially air-tight.

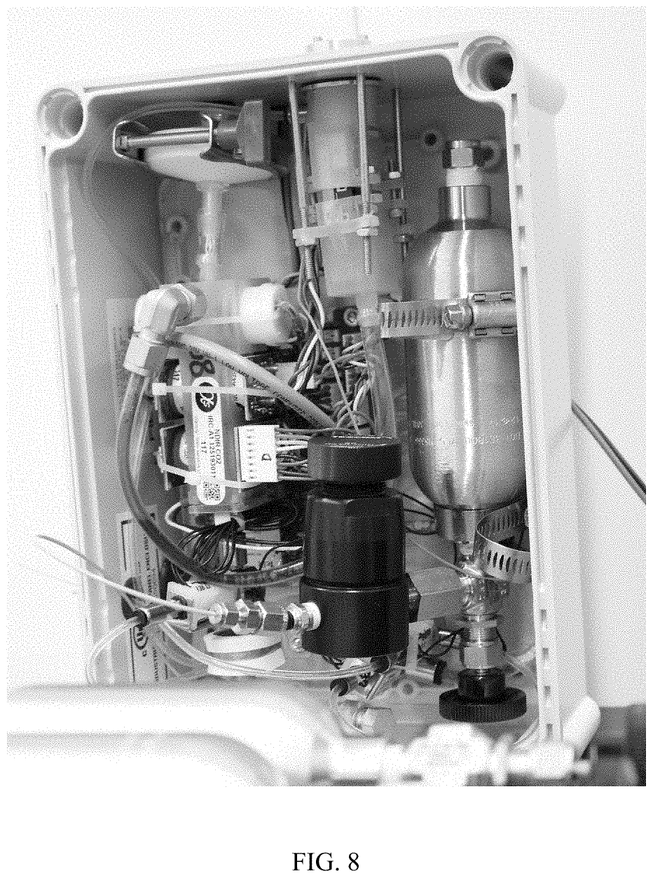

An alternative stationary multi-pollutant monitor is shown in FIG. 8. The main distinguishing characteristic of the monitor of FIG. 8 is the addition of a gas cylinder and its delivery system for use in checking the known concentration response of the sensors of the monitor or the zero response if a sensor is not responsive to any of the chemical components in the standard cylinder. The monitor of FIG. 7 does not use a gas cylinder for calibration.

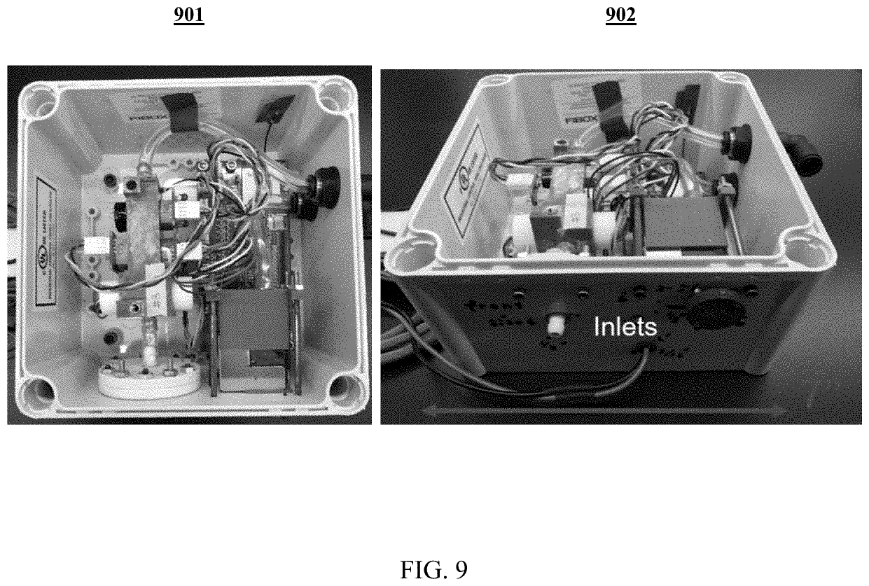

Still another exemplary prototype multi-pollutant monitor is shown in FIG. 9, which comprises photographs 901 and 902. The monitor of FIG. 9 has a small form factor, and is shaped as a square measuring seven inches on each side and with a height of 5 inches. In some embodiments, a smaller form factor may be achieved by removing the calibration system.

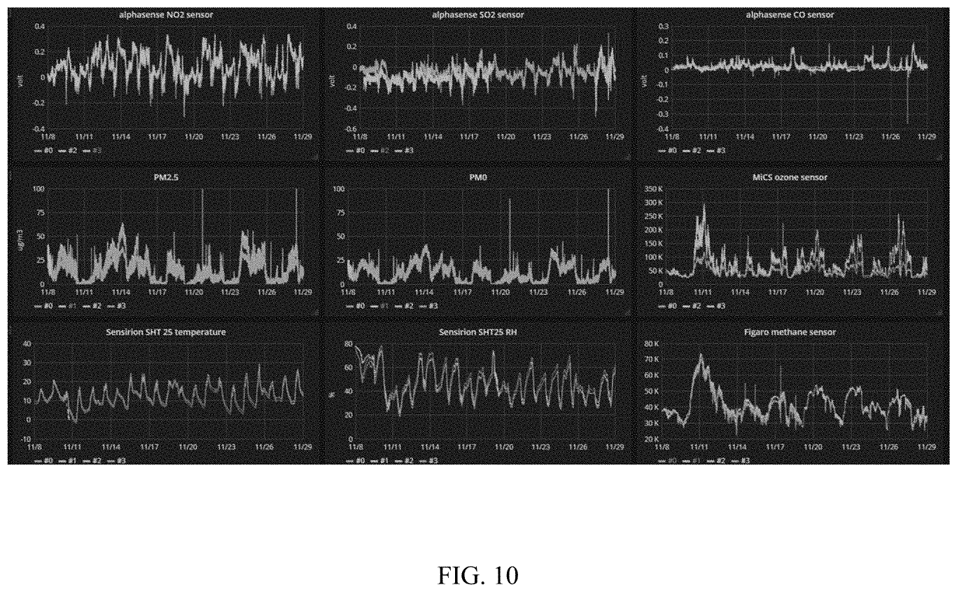

In some embodiments, a multipollutant monitor of the present invention may include a real-time monitoring interface, for example the interface shown in FIG. 10. The interface shown in FIG. 10, or a similar interface, may be accessed via a network connection, for example via an HTTP or HTTPS connection from a wired or wireless network connection to a controller in the monitor. The data may be presented as one graph per pollutant, or may combine multiple pollutants on a single graph. In some embodiments, the interface updates in real-time, while in other embodiments, for example to save power, the interface may update at a lower frequency or only on demand. In some embodiments, a network-connected real-time monitoring interface may also include one or more remote control functions, including, but not limited to running a calibration routine, enabling or disabling particular sensors, or turning monitoring on or off.

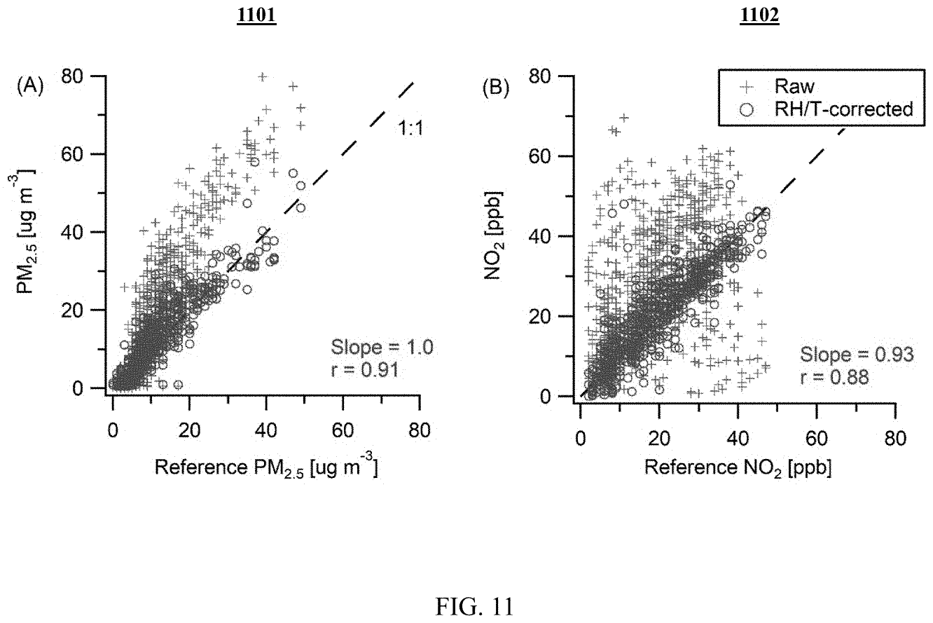

In one embodiment, a calibration method or system of the present invention may calculate a response factor with regard to one or more pollutants being measured. For example, sensor signals may change at different rates or with different curve shapes in response to different pollutants. In one embodiment, calibration measurements using various sensors are made under a variety of different temperature and humidity conditions in order to produce a more accurate calibration model for sensors of pollution measurement devices. An example of calibrated and RH/T-corrected data is shown in FIG. 11. Certain embodiments of the present invention may further refine sensor calibrations using a secondary response from a second sensor. For example, in one embodiment a first sensor for monitoring a first compound is fluidly connected to a second sensor in the manifold for monitoring a second compound. The second sensor has a known secondary response to the first compound in addition to its primary response to the second compound. In some embodiments, the calculated secondary response from the second sensor to the first compound may be used to refine or calibrate measurements taken by the first sensor. In one example, the second sensor is an CO sensor and the first sensor is a NO₂ sensor, and the second sensor is known to also be responsive to NO₂.

In some embodiments, a PM sensor includes a separate zeroing system comprising a pump or valve configured to pressurize the PM sensor inlet channel, flushing out any debris that might affect the results.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the system and method of the present invention. The following working examples therefore, specifically point out the exemplary embodiments of the present invention, and are not to be construed as limiting in any way the remainder of the disclosure.

A suite of sensors was built into a multipollutant monitor to measure the concentrations of carbon monoxide (CO), nitrogen dioxide (NO₂), nitric oxide (NO), sulfur dioxide (SO₂), carbon dioxide (CO₂), methane (CH₄), ozone (O₃), and particulate matter (PM), as well as temperature and relative humidity to correct for their influences on sensor responses during field deployment. A survey of available commercial sensor technology was conducted, the best performing sensors were selected to integrate into a fully customized electrical and physical system. One exemplary embodiment of a monitor contains an NO sensor and the remaining seven sensors, while another exemplary embodiment of a monitor contains an SO₂ sensor and the remaining seven sensors. Monitors containing SO₂ sensors may be used for example in applications related to emissions from coal burning. Monitors containing NO sensors, together with measurements from the CO, CO₂, NO₂, O₃ and PM sensors, will provide more insights into traffic emissions and the related photochemical processes. A CH₄ sensor is incorporated for future studies involving methane emissions and compliance, and along with the CO₂ sensor, can be used to evaluate greenhouse gas emissions.

4-electrode electrochemical CO, NO₂, NO and SO₂ sensors from Alphasense (http://www.alphasense.com) were chosen for the multipollutant monitor. Different models of electrochemical sensors manufactured by Alphasense have been tested and have shown great promise to perform measurements in an urban ambient environment. The 4-electrode configurations were chosen over the 3-electrode sensors, because the extra auxiliary electrode (AE), with the same build as the working electrode (WE) but not exposed to the analyte, provides a background electrode response and reduces the influences of temperature, relative humidity (RH), and pressure on sensor signals. The 4-electrode electrochemical sensors have two forms, A4 and B4, both designed for environmental monitoring at parts-per-billion (PPB) level. According to the Alphasense product specifications, the CO, NO₂, and NO sensors in the B4 series have sensitivities 80%, 35%, and 50% higher than their A4 counterparts, and on par with the SO₂ sensor. However, the B4 sensors are approximately four times the size of the A4 sensors. Despite their lower sensitivities, the compact A4 series electrochemical sensors were chosen for the multipollutant monitor to minimize the overall size of the device.

A CO₂ measurement was performed with the Alphasense NDIR sensor, which has an estimated detection limit of 1 ppm. The NDIR sensor has a broadband light source, and two bandpass filters centered at 4.26 μm and 3.95 μm. The 4.26 μm filter coincides with the CO₂ absorption band centered at 4.2 μm. The 3.95 μm light is not absorbed by CO₂, and works as a reference to account for potential drift in light intensity caused by lamp aging and power supply change. The CO₂ sensor has similar dimensions as the A4 electrochemical sensors.

The Figaro TGS2600 gas sensor was chosen to measure methane concentrations. The manufacturer's specification suggests that this sensor is also sensitive to analytes such as CO, hydrogen (H₂), and volatile organic compounds (VOC), including ethanol and isobutane. The cross sensitivity from CO can be corrected by CO measurement from the onboard CO sensor. The VOC effect can be resolved by adding one or more layers of charcoal cloth on top of the sensor to remove VOC. The VOC effect was removed by adding one layer of hydrocarbon cloth on top of the sensor to adsorb and block VOC. After continuous exposure to laboratory room air VOC for one month, the hydrocarbon cloth could still effectively remove ethanol vapor, when an open ethanol vial was placed in front of the sensor, with no signal changes observed.

The MiCS-2614 sensor was chosen for O₃ measurement because of its low cost and small size (5 mm×7 mm×1.55 mm). Previous studies found the MiCS sensor agreed with 2B Technologies ozone monitor in the ozone concentration range from 20 ppb to 100 ppb, with over-measurement under 20 ppb and significant under-measurement above 100 ppb by the MiCS sensor. The Alphasense Ox sensor can also be used here.

Particulate matter is measured with a miniature PM sensor PMS A003 (35 mm×38 mm×11.8 mm), made by Plantower (http://www.plantower.com). The sensor has an internal laser and uses scattered light to differentiate sizes and count particles. The device reports mass densities in PM1, PM2.5, and PM10 with precision of 1 μg/m³, as well as particle number densities for particle sizes larger than 0.3 μm, 0.5 μm, 1 μm, 2.5 μm, 5 μm, and 10 μm. Prior studies suggested that earlier versions of this sensor, PMS 1003 and PMS 3003, tested in laboratory and ambient environments, have correlation coefficients of measurements ranging from 0.7 to 0.93 between the PMS sensors and reference instruments.

The electrical system for one embodiment of a multipollutant monitor was designed to have modularized functions on individual circuit boards. Each sensor has designated analog circuitry to supply power, amplify signals, and filter electronic noise. The analog signals are fed to onboard analog-to-digital converters (ADC), and only digital data are transmitted from the sensor boards to the microcontroller to avoid noise pick-up by the wires.

The Alphasense electrochemical sensors were powered with potentiostatic circuitries with zero bias for the CO, NO₂, and SO₂ sensors, and a 200-mV bias for the NO sensor. Special care was taken to match the input impedance for the NO potentiostatic circuit to minimize noise. The circuit amplification was designed to output an analog signal of approximately 1 volt for 100 ppb NO, SO₂, NO₂, and 10 ppm CO, but can be adjusted for other environments/conditions. Each board has one analog-to-digital converter (ADC) on board, and only digital data are transmitted from the sensor boards. The onboard ADC sequentially converts the amplified and filtered signals generated by the auxiliary electrode (AE) and the working electrode (WE). The AE voltage was recorded as the background signal, and the differential signal between WE and AE voltages was used as the sensor signal for calibration and measurement purposes. The final dimensions of the electrochemical sensor circuit boards in one embodiment are 24 mm×36 mm.

The CO₂ sensor was driven with a 2 Hz 5V 50% duty cycle waveform clocked by a MEMS (Micro-Electro-Mechanical Systems) oscillator. The outputs of the CO₂ sensor are two DC-biased sinusoidal waves from the reference and active channels, and subsequent circuitries were implemented to remove the DC offset and amplify the sinusoidal signals. Two peak detection circuits were applied to sample and hold the peak heights of the two amplified sinusoidal waves to be read sequentially by the ADC. This design used significantly less processing resources, in comparison with continuous sampling and peak detection through software.

The CH₄ and O₃ sensors and support circuitry were placed a single circuit board to save space and accommodate mechanical requirements. The CH₄ and O₃ sensors work by changing their resistances when exposed to their corresponding analytes. Hence, voltage dividers with low temperature coefficient load resistors were applied, and the sensor resistances were derived by sampling the voltages across the load resistors through ADCs. The final size of the CH₄—O₃ board is 15 mm×15 mm.

The humidity/temperature (RH/T) sensor was placed on a separate small circuit board (8 mm×9 mm), so that the sensor's temperature measurement is not affected by heat generated by voltage drop across circuit board traces in the presence of other components. The RH/T sensor and the PM sensor both output digital signals, and the signals are acquired by the microcontroller directly.

A central control board is configured to step up or down input voltages, power on or off components, and read, process, store and transmit sensor data. The control processes are achieved with Cypress 68 pin PSoC 5Ip microcontroller, which interfaces with sensors through digital communication peripherals (I²C and UART). The data acquisition frequencies were set as follows: The NO₂, NO, SO₂, CO sensors were sampled every 160 ms, with AE and WE signals each taking up 80 ms sequentially. The CH₄ and O₃ sensors were also sampled every 160 ms, and they both had only one signal channel. The RH/T sensor was sampled every 160 ms for either RH data or temperature data sequentially, making their actual sampling period 320 ms. The CO₂ sensor was sampled with 2 Hz frequency in accordance with the input drive frequency, for both the active and reference channels. The PM sensor was sampled every 640 ms, to accommodate its low data output rate.

All the acquired sensor data were written to an SD card, and every 10.24 s the data averaged over the past 10.24 s were transmitted to a database hosted on a cloud server through an onboard 4G cellular module. Both ADC conversion and cellular communication involve significant waiting time ranging from a few milliseconds to one second. To achieve fast and continuous sensor data collection while maintaining simultaneous cellular data transmission, a task scheduler was designed within the microcontroller software to track the status of the sensors and cellular module, and at preset time intervals service the components with processes such as read, write, send and receive. The data stream stored on the SD card and the cloud server include: reference and differential signals for the electrochemical sensors and the CO₂ sensor, resistances of the CH₄ and the O₃ sensors, relative humidity, temperature, PK, PM2.5, PM10, particle number densities for particle diameter above 0.3 μm, 0.5 μm, 1 μm, 2.5 μm, 5 μm, and 10 μm, plus input power voltage and microcontroller die temperature. The last two items are system parameters to check for normal operating conditions. Besides servicing sensors, the control board also activates solenoid valves periodically to perform calibration and background measurement, and powers a piezoelectric blower to circulate ambient air for the gas sensors.

### Instrument Design and Testing

The electrical system for the multipollutant monitor was designed to have modularized functions on individual circuit boards. Each sensor has its designated circuitry to power the sensor and generate analog sensor signals, which are fed to on-board analog-to-digital converters, and the digital signals are acquired and processed by a microcontroller. Transmitting digital instead of analog sensor signals can better protect signal integrity against noise interference along the data wires. Sensor data received by the microcontroller are relayed to a cellular module and transmitted to a database hosted on a cloud server for online data visualization.

The overall system design for the purposes of the experiment followed the simplified electronics and flow diagram in FIG. 6. The electronic circuits for CO and SO₂ and NO₂ sensors were designed and tested to measure their corresponding analytes at 0-100 ppb, 0-100 ppb, and 0-10 ppm dynamic ranges. The designed circuit boards have dimensions (24 mm×36 mm) comparable to the sensor nodes (20 mm in diameter), so the boards do not take up significant extra space in the assembly of the final device. The methane, ozone, and humidity/temperature sensors all have compact dimensions. They were placed on two separate circuit boards, with humidity/temperature sensor standing alone, because laboratory and field tests suggest proximity to other sensors and circuits causes the temperature measurement to bias high. The particulate matter (PM) sensor outputs digital signals, and wires are directly soldered to the miniature connector mating with the sensor socket. No external circuitry was applied for the PM sensor to conserve space.

The gas sensors were calibrated and tested to determine linearity, dynamic range, and detection limits. The sensitivities and zero-concentration offsets can vary among sensors, and calibrations on individual sensors are necessary to ensure accurate quantitative results. For the methane sensor, its resistance changes nonlinearly with methane concentrations, and with its current noise level, it can distinguish changes of 0.1 ppm near ambient methane concentrations. The methane sensor is known to be responsive toward ethanol and isobutane. Hence, a hydrocarbon cloth was added to the sensor to filter out these organic components in the atmosphere. When the methane sensor was placed in a chamber filled with 2% ethanol, its signal did not change until the hydrocarbon cloth was removed.

In one embodiment of a multi-pollutant monitor system as contemplated herein, a sampling manifold was designed to support the gas sensors and isolate their sensing areas from the rest of the device. The manifold was 3D printed with WaterShed XC, which had gas-tight finish. O-rings were used to seal and secure sensors to the manifold. To minimize potential ozone loss, the ozone sensor is positioned close to the manifold inlet. The outlet of the manifold is connected to the piezoelectric blower. The internal volume of the manifold is approximately 9 ml.

The ambient air entering the manifold is passed through a 2 μm 47 mm Teflon membrane to remove particles and keep the inside of the manifold clean. The filter holder consists of two machined Teflon parts that are designed with the geometry to fit a commercial KF 40 clamp for compression. The ambient air exiting the filter holder flows through a Teflon liner to enter the manifold, which minimizes potential loss of ozone to the printed material.

The CH₄ sensor inside the manifold is covered with a layer of charcoal cloth, which is secured by a 3D-printed ABS cylindrical shell. This charcoal cloth layer is configured to filter out VOC interference for the sensor. For instance, when covered by the charcoal cloth, the CH₄ sensor does not respond to ethanol concentrations as high as 2%. The resistance to ethanol persists even after continuous exposure to outdoor VOC for 3 months. When the CH₄ sensor covered with the used charcoal cloth was placed directly above an open vial of ethanol, sensor resistance dropped by approximately 5 kΩ, which is equivalent to 0.3 ppm methane. In an ambient environment, however, such highly concentrated VOC vapors are unlikely to be encountered. Despite the resilience and effectiveness of the charcoal cloth, a good maintenance practice calls for quarterly replacement.

Inlet and outlet enclosures were designed for the PM sensor to direct the air flow. Specifically, the inlet enclosure contained a 3D-printed plastic holder to support the sensor and an aluminum duct, through which air would flow into the sensor inlet. Aluminum was chosen over 3D-printed plastic material as the inlet duct, to avoid the buildup of static charges on plastic surface that could deflect particles. The front of the aluminum duct was covered with an aluminum disk placed ⅛″ above it, between which a 32×32 mesh stainless steel wire cloth was installed to block insects and large dust particles. When the PM sensor was powered up, ambient air would flow around the disk, pass through the wire cloth, and enter the aluminum duct and the sensor inlet. The aluminum disk was placed above the inlet to block sunlight and other direct light, which was shown to interfere with normal operation and cause the sensor to output PM mass concentrations above 3000 μg/m³.

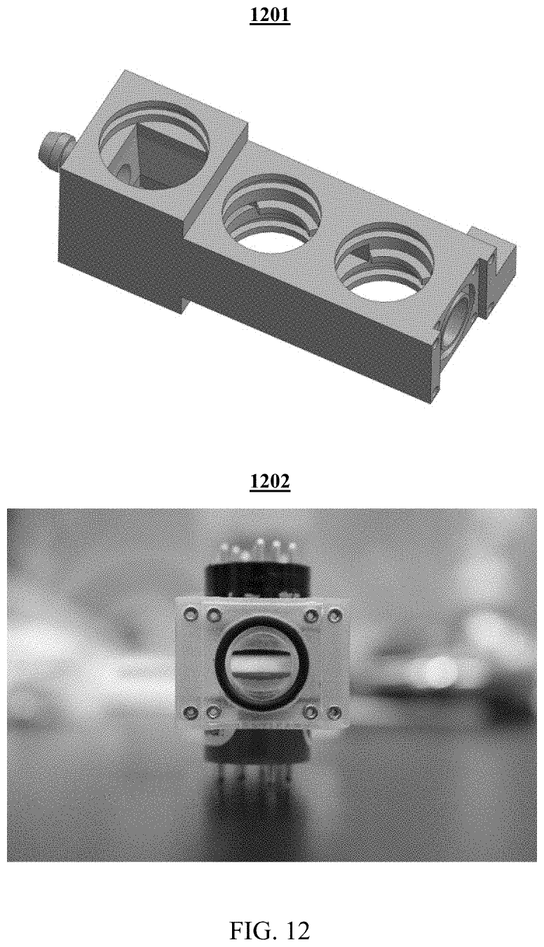

In another embodiment of a multi-pollutant monitor as contemplated herein, Gas sensors are mounted in a manifold as shown in FIG. 12. 1201 is a 3D model of an exemplary manifold, and 1202 is a photograph of an exemplary manifold with sensors mounted therein, according to the 3D model 1201. In the manifold of FIG. 12, air was actively pumped through by a micro piezo blower installed at its end. The manifold has internal volume of approximately 10 ml, which reduces the air residence time to 2 s with an inlet flow rate of 0.3 SLPM. This fast exchange rate will ensure minimal sample loss and fast sensor response toward environmental changes. A filter holder was installed in front of the manifold to remove PM, and keep the manifold clean. The PM sensor has a separate inlet placed along the gas sampling manifold. The sensors and electronics assembly are shown in the system diagrams in FIG. 6. A brief road side test with the multipollutant monitor in a downtown area captured elevated CO and PM concentration from the traffic. The system collects data for NO₂, SO₂, CO, methane, ozone, humidity, temperature with 0.2 s frequency, and data for PM with 1 s frequency. The microcontroller collects the sensor data for 10 s and sends the 10 s average to a cloud server. At the same time, a copy of all the original sensor data is preserved on an SD card. An online platform was laid out to visualize the data through PC and smartphone web browsers, and scripts have been developed to download the data from the server for further analysis.

### Calibration and Zero System

To improve data quality during field deployment and to better characterize sensor performance, a calibration and zero system was designed and tested.

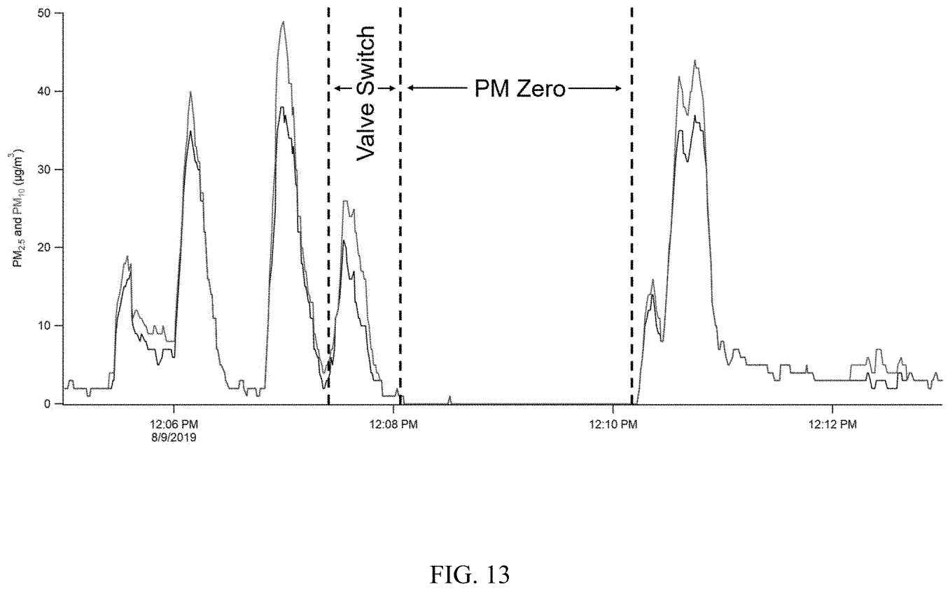

The exhaust from a piezoelectric blower, in which particles had been filtered out by a Teflon membrane, was directed to the aluminum inlet of the PM sensor to check its baseline zero signal. A graph showing the effect of the PM sensor zeroing process is shown in FIG. 13. As shown, the PM2.5 and PM₁₀ levels both receded to zero in response to the blower clearing the inlet and providing particle-free air flow to the sensor. The inlet may be flushed in one embodiment with filtered exhaust from the gas measurement system.

To obtain the zero-concentration signal of the various gas sensors, a series of scrubbing materials were tested to remove gas-phase analytes. In one exemplary embodiment, soda lime, steel wool, and activated carbon were chosen because of their efficacy at removing CO₂, O₃ and NO₂. To obtain the zero-concentration signals of the NO₂, CO₂ and O₃ sensors, the exhaust of the piezoelectric blower was passed through packed soda lime and directed to the gas sensors through a side port on the manifold near the inlet. The flow rate through the packed tube was 50-400 sccm. With the 9 ml internal volume, the air inside the manifold was re-circulated and passed through the packed tube to achieve efficient analyte removal.

A gas delivery system was designed to fill the manifold with known concentrations of gas standards to evaluate drifts in sensor sensitivities across time. A miniature gas cylinder (2″ OD×5.5″) was used. The main valve and pressure regulator were adjusted to deliver 30 sccm standard gas flow into the manifold through the exhaust port of the piezoelectric blower with the blower off. It took approximately 1 min or less for the relevant sensor signals to stabilize.

A water permeation setup was added to the standard gas delivery line to maintain the humidity inside the manifold and to prevent the sensors from drying out. The water permeation device was built by fitting a Teflon film between the end of a Teflon tubing filled with water and a Swagelok tube connector. The thin film of the Teflon material helped to contain the water and prevent leakage. Water vapor can permeate through the Teflon film to increase the RH of the standard gas.

Three 3-way solenoid valves were placed in the system to alternate the sampling scenario among normal ambient sampling, PM zero, gas zero, and gas calibration.

### Results

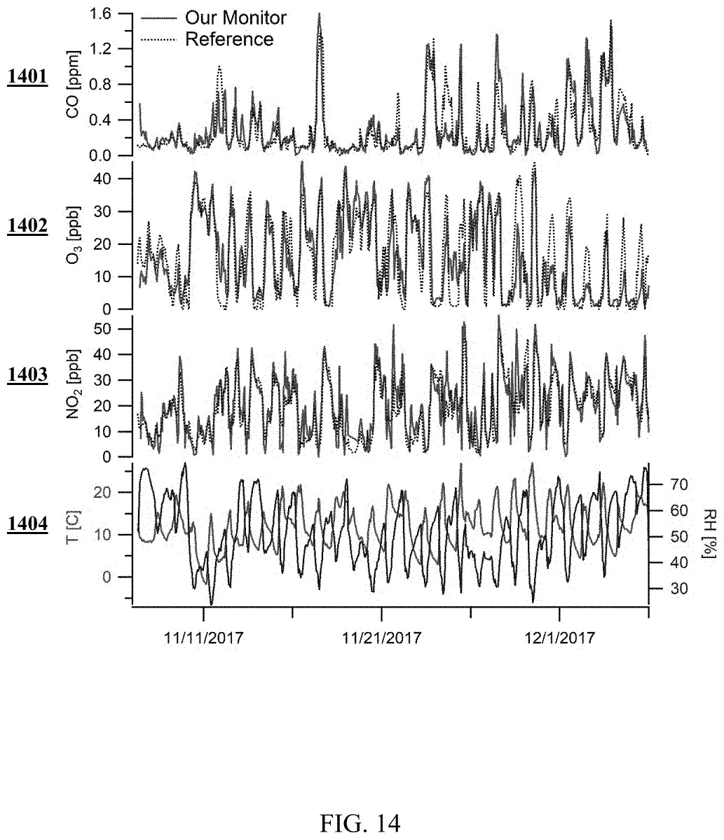

Data were collected by a stationary multi-pollutant monitor in an outdoor field trial in Baltimore, Md. The measurements were taken at the Old Town reference measurement site with data collected from the monitor at least every 10 s. Graphs of resulting data are shown in FIG. 14, including graphs 1401-1404. Graph 1401 shows measured CO concentration (solid red line) over time along with a regional EPA reference measurement (broken line) during the same time period. Graph 1402 shows measured Ozone concentration (solid red line) over time along with a regional EPA reference measurement (broken line) during the same time period. Graph 1403 shows measured NO₂ concentration (solid red line) over time along with an EPA reference measurement (broken line) during the same time period. The final graph 1404 shows temperature in degrees Celsius (red) and relative humidity (blue) over the same time period as graphs 1401-1403. FIG. 11 also contains data from this trial.

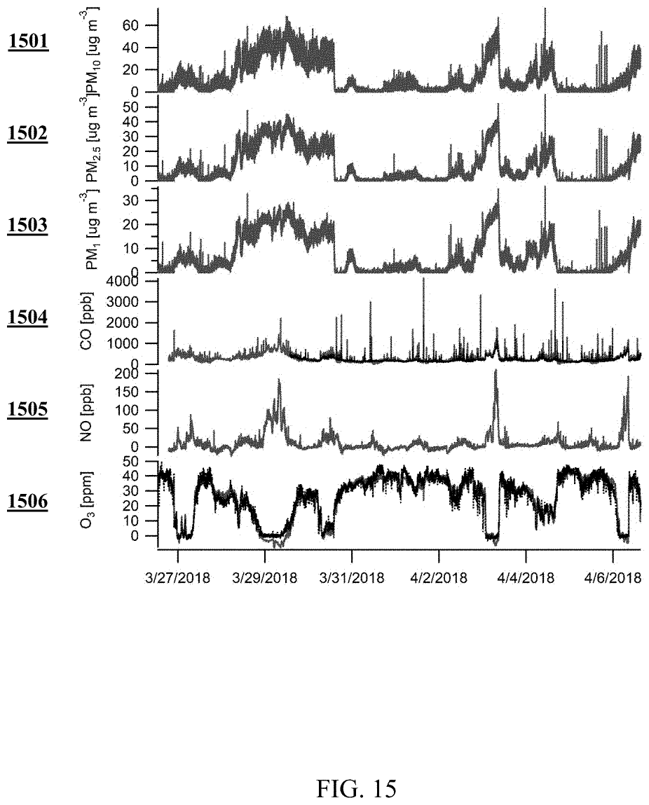

Additional data was collected in an outdoor field trial in New Haven, Conn., shown in graphs 1501-1506 in FIG. 15. Data was collected at 10 s intervals or faster. Graph 1501 shows PM10 concentration over time, graph 1502 shows PM2.5 concentration over time, and graph 1503 shows PM1 concentration over time. Graph 1504 shows carbon monoxide concentration over time (red) and reference measurements (black). Graph 1505 shows NO concentration over time, and graph 1506 shows ozone concentration over time as measured (red) and a reference measurement (black).

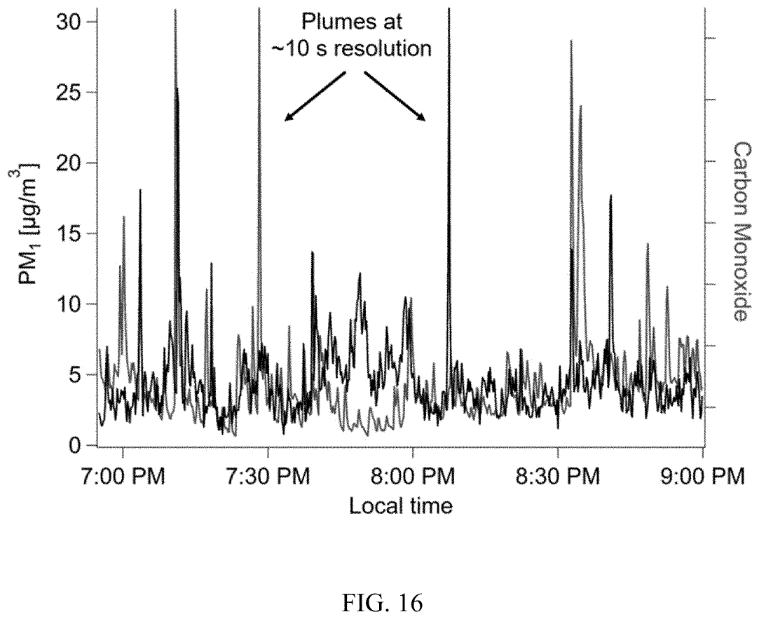

With reference now to FIG. 16, a graph of data collected from a stationary multi-pollutant monitor positioned next to a road is shown. The data indicates high time resolution (approximately 10 s) plumes, where the multi-pollutant monitor captured elevated CO (red) and PM1 (black) concentrations at the passage of high-emission vehicles.

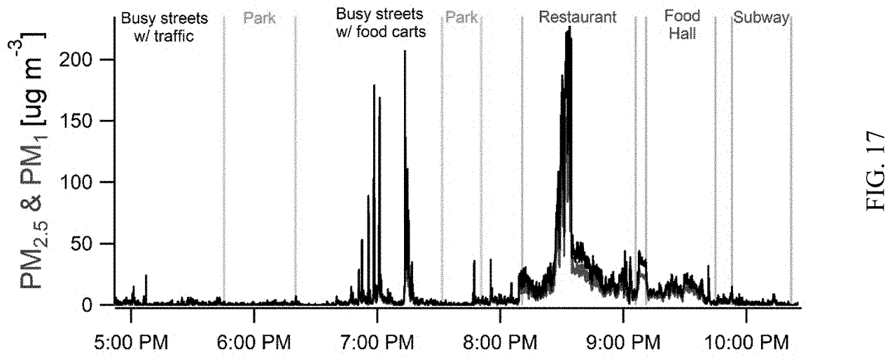

Exemplary data collected from a personal, portable multi-pollutant monitor is shown in FIG. 17. The data is shown graphed over time, annotated along the top with the locations in which the data was collected, as the user traveled around Manhattan (New York City). The graph shows measured PM1 concentration in red, and measured PM2.5 concentration in black.

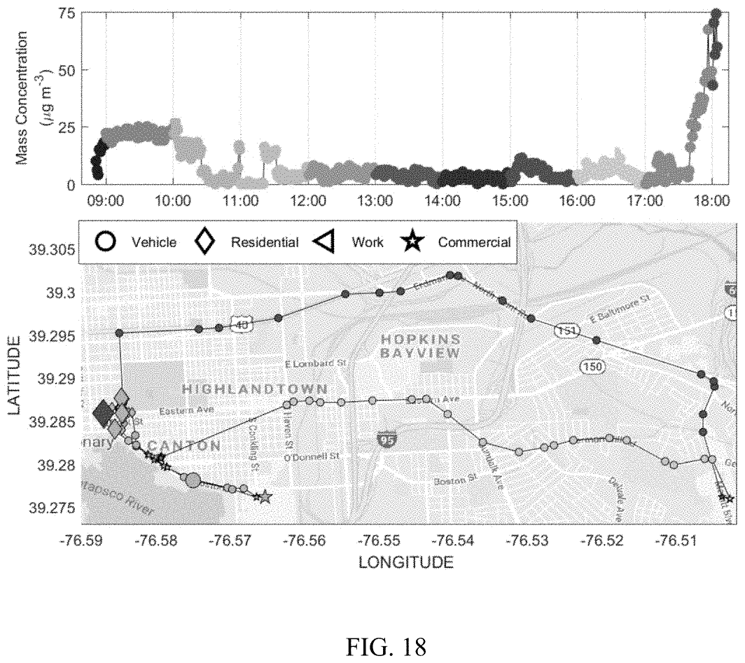

Exemplary data from another personal, portable multi-pollutant monitor trial is shown in FIG. 18, which includes a map indicating the path taken by the user of the portable monitor in Baltimore, Md., along with a measured mass concentration of PM2.5 over time during the path.

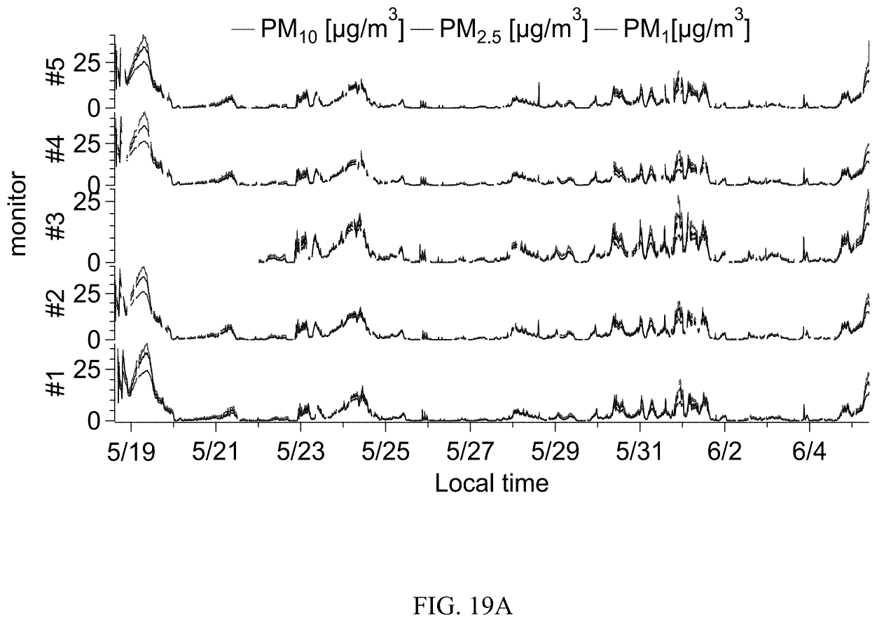

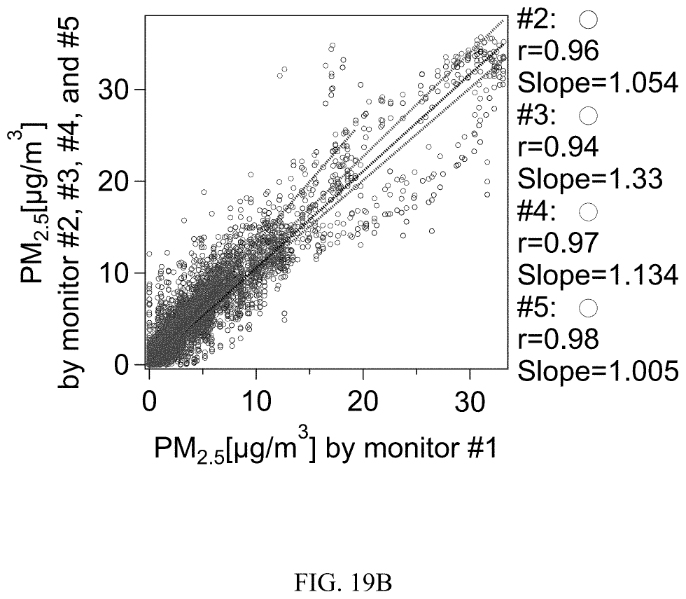

With reference now to FIG. 19A and FIG. 19B, comparison data are shown across multiple multipollutant monitors demonstrating that the monitors are consistent in their measurements. Five monitors were placed near each other and measurements taken over an 18-day period at ten minute intervals. PM1, PM2.5, and PM10 levels were measured and the results compared. The resulting correlation coefficients are shown in FIG. 19B. Across the five monitors, the correlation coefficients varied between 0.94 and 0.98.

### Sensor Response Time

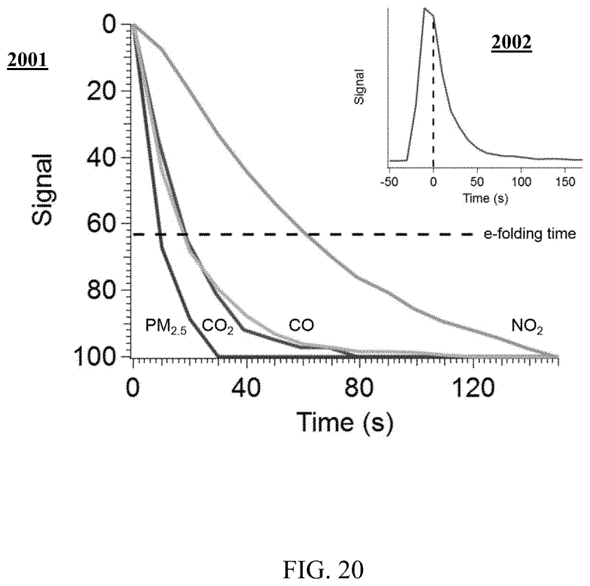

A graph of sensor response times is shown in FIG. 20 that have been reduced through the use of high sampling flow rates and the minimized internal volume of the sensor manifold. The x-axis of the graph 2001 shows the time in seconds, while the y-axis shows the measured concentration of various particulate and gaseous pollutants. The figure demonstrates the changes in concentration after the end of a pollutant plume, observed outdoors. The inset graph 2002 shows an example plume, i.e. the concentration of gas released into the environment being measured by the various sensors. As shown, the response time to a drop in gas concentration is on the order of seconds to tens of seconds, dependent on the sensor.

### Calibration Results

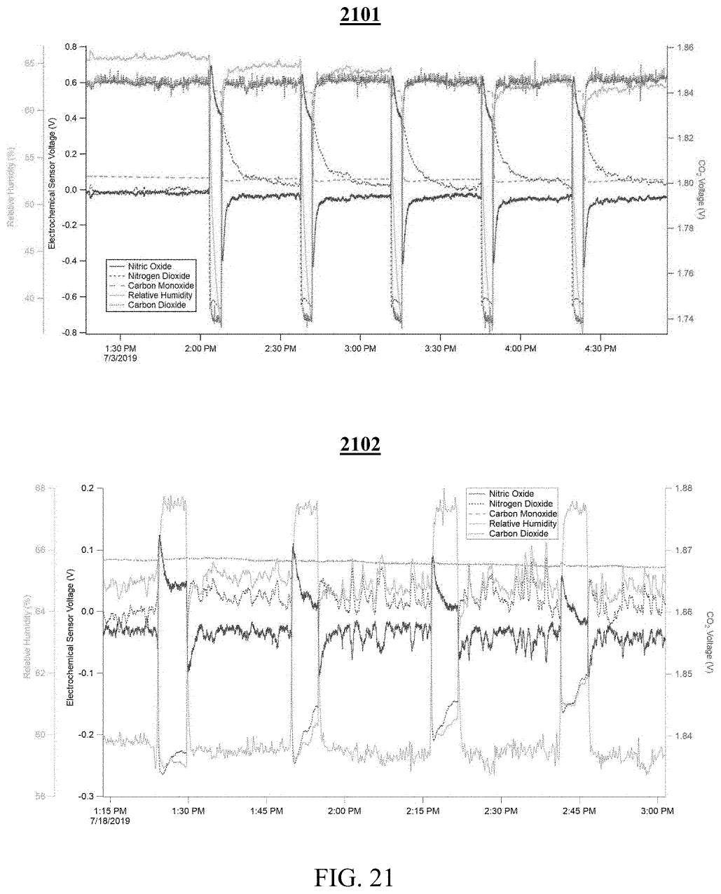

Graphs showing the performance of the online calibration system and features within the cylinder units are shown in FIG. 21.

Graph 2101 shows the repeatability of the gas standard calibration of 2000 ppm carbon dioxide and 5 ppm carbon monoxide through five runs. The x-axis shows time in seconds, while the y-axes variously show the relative humidity (yellow) and the electrochemical sensor voltages. These calibrations with the gas cylinder also act as zeros for NO and NO₂. Graph 2102 shows the ability of the zero-trap system to scavenge NO₂, and CO₂ using activated carbon, soda lime, and stainless steel wool, respectively. As expected, CO does not respond to the current formulation of the zero trap and is shown here to demonstrate the consistency in its concentration over the experiment. Note that the CO₂ voltage response is inversely proportional to sampled concentration in both graphs 2101 and 2012, and NO and NO₂ signal changes during and between each calibration period are due to changes in RH. The x-axis shows time, while the y-axes variously show the relative humidity (yellow) and the electrochemical sensor voltages.

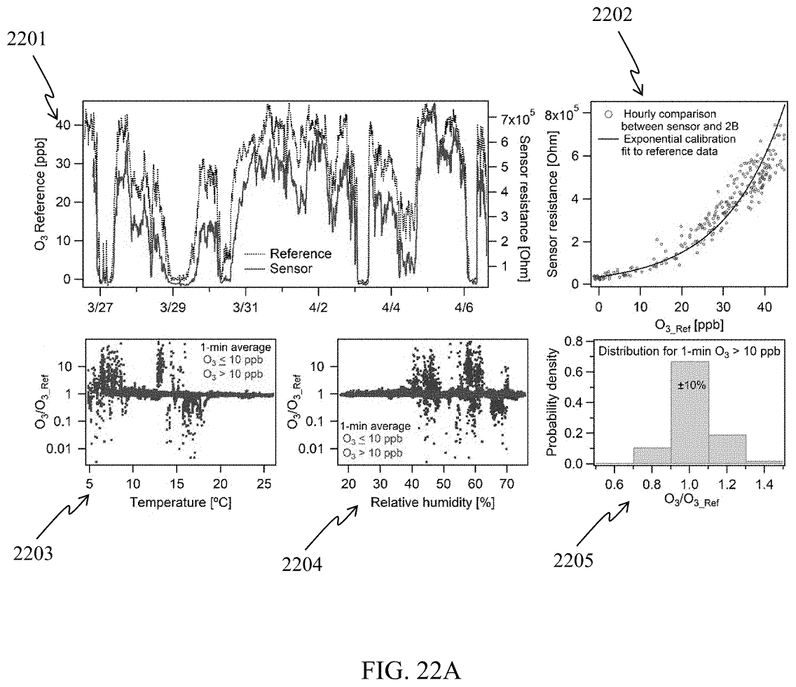

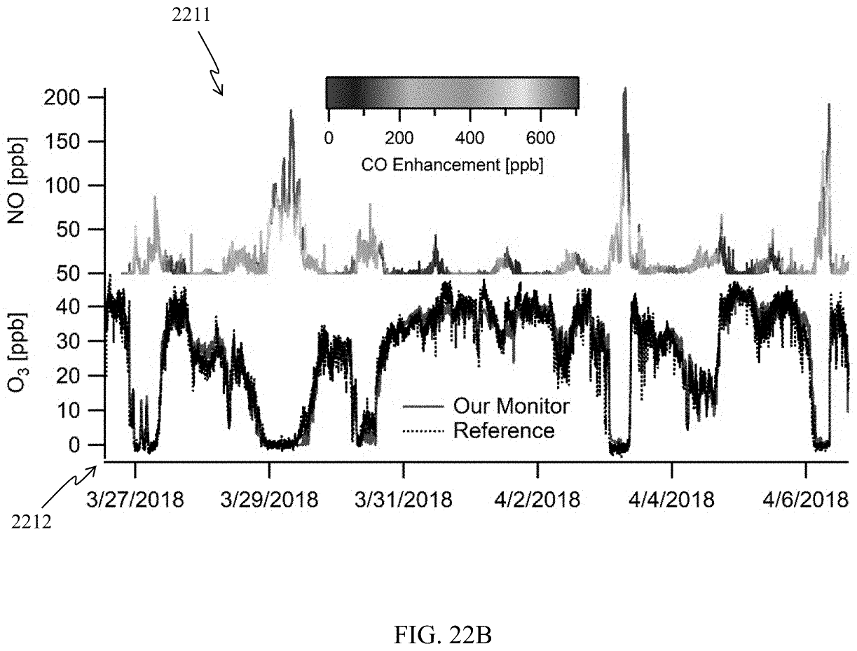

Results of an ozone calibration experiment conducted over two weeks in New Haven, Conn. are shown in FIG. 22A. Graph 2201 shows the measured ozone concentration (red) and a reference sensor (black). Graph 2202 shows a comparison of raw sensor signal against a 2-B Tech reference monitor. Graphs 2203 and 2204 show the ratio of the calibrated sensor of a disclosed multi-pollutant monitor vs. a reference measurement for concentrations greater and less than 10 ppb over the range of relative humidity and temperatures observed, with no dependence on humidity and a slight temperature dependence. At concentrations greater than 10 ppb the measurements of the multi-pollutant monitor are much more accurate, with 70% of the 1-min average data falling within ±10% of the reference. Graph 2205 shows a probability density diagram of the difference between the measured data and the reference data for greater than 10 ppb.

As shown in FIG. 22B, Roadside NO and O₃ data from the New Haven, Conn. experiment shows large NO plumes (graph 2211). The presence of NO is confirmed by both the titration of O₃ (i.e. NO+O₃ reaction) to zero (see graph 2212) and large enhancements in CO (a combustion co-pollutant). It is noteworthy that the NO sensor used does not have significant cross-response to O₃ or CO.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. An air quality measurement device comprising:
a housing configured to rest on a shoulder of a user comprising an air inlet directed to a breathing zone of the user, and an air outlet;
an air quality sensor and an air pump within the housing and connected in-line between the air inlet and the air outlet.

2. The air quality measurement device of claim 1, wherein the housing comprises a form-fitting crescent shape.

3. The air quality measurement device of claim 1, wherein the housing comprises an attachment mechanism configured to attached to a shoulder area of apparel or a shoulder strap.

4. The air quality measurement device of claim 3, wherein the shoulder strap is one of a bag strap, a backpack strap and a harness strap.

5. The air quality measurement device of claim 1, wherein the air inlet is directed towards an area in front of a user's face and the air outlet is directed to an area behind the user.

6. The air quality measurement device of claim 1, wherein the air quality sensor is at least one of a gas sensor and a particulate matter sensor.

7. The air quality measurement device of claim 1 further comprising a connection to an external battery and an external cell board.

8. The air quality measurement device of claim 1 further comprising an internally contained battery and an internally contained cell board.

9. An air quality measurement system comprising:
the device of claim 8; and
a battery and cell board unit configured to attach to a user using an attachment mechanism separate from the housing.

10. A calibration system for an air quality measurement device comprising:
a gas sensor, a particulate matter sensor, and particulate matter zeroing element configured to calibrate the particulate matter sensor.

11. The calibration system of claim 10, wherein an outlet to a manifold housing the gas sensor and filtered of particles at a main inlet is connected to the particulate matter zeroing element.

12. The calibration system of claim 11 further comprising:
a 3-way valve configured to switch airflow from the outlet between the particulate matter zeroing element and a particulate matter sensor inlet.

13. The calibration system of claim 10, further comprising a gas phase zeroing element comprising a packed bed of mixed catalysts and adsorbents configured to filter out pollutants.

14. The calibration system of claim 13, wherein the packed bed comprises at least one of soda lime, ascarite, activated carbon, molecular sieves and steel wool.

15. The calibration system of claim 13, wherein the packed bed comprises at least two of soda lime, ascarite, activated carbon, molecular sieves and steel wool.

16. The calibration system of claim 13, wherein the packed bed comprises at least three of soda lime, ascarite, activated carbon, molecular sieves and steel wool.

17. The calibration system of claim 13, wherein the packed bed comprises soda lime, ascarite, activated carbon, molecular sieves and steel wool.

18. The calibration system of claim 13, wherein the gas phase zeroing element comprises a cylinder comprising pure air.

19. The calibration system of claim 13, wherein the gas phase zeroing element comprises a cylinder comprising a gas standard that does not contain any gases the gas sensor is responsive to.

20. The calibration system of claim 13, wherein the gas phase zeroing element comprises a UV-generating lamp configured to generate a constant concentration of ozone.

21. The calibration system of claim 11, wherein the system is configured to provide zero calibration measurements across a range of relative humidity and temperature points.

22. The calibration system of claim 21, further comprising a water vapor permeation device configured to maintain at least a minimum relative humidity inside the manifold during a gas sensor calibration.

23. The calibration system of claim 10, wherein the system is configured to provide air quality measurement for a plurality of pollutants.

24. The calibration system of claim 10, further comprising a gas phase calibration system comprising:
a gas cylinder, the gas cylinder containing a known gas mixture; and
a controlled dispensing system configured to expose the gas sensor to a fixed quantity of the known gas mixture;
wherein the gas sensor is responsive to the known gas mixture directly as a measured analyte of interest or indirectly as a cross-responsive gas.
